(19) United States
(12) Patent Application Publication
(10) Pub. No.: US 2006/0234222 A1
(43) Pub. Date: Oct. 19, 2006

(54) Soluble recombinant protein production

(76) Inventors: Brendan McKeown, Ballyciare (GB); Christopher Scott, Belfast (GB); Alan McBride, Salvador (BR); Richard Buick, Ballymena (GB); Jim Johnston, Belfast (GB)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(21) Appl. No.: 10/501,357

(22) PCT Filed: Dec. 30, 2002
(86) PCT No.: PCT/GB02/05941

§ 371 Date: Jun. 9, 2006

(30) Foreign Application Priority Data
Dec. 28, 2001 (GB) 0131026.7
Dec. 28, 2002 (GB) 0230247.9

(51) Int. Cl. C12Q 1/68; C07H 21/04; C12P 21/06; C07K 14/195; C12N 1/21; C12N 15/74

(52) U.S. Cl. 435/6; 435/69.1; 435/252.33; 435/488; 530/350; 536/23.7

(57) ABSTRACT

Described is a method of producing a soluble bioactive domain of a protein, the method comprising the step of selecting suitable soluble subunits of a protein and assessing the produced protein for desired activity. The method may comprise the steps of amplifying DNA encoding at least one candidate soluble domain, cloning the amplified DNA into at least one expression vector, using each of said vectors into which the DNA has been cloned to each transfect or transform one or more host cell strains, expressing said DNA in one or more host cell strains, and analyzing expression products from said host cells for solubility.

The present invention relates to methods of producing proteins, in particular to methods suitable for high-throughput production of soluble proteins.

This application describes a methodology for the rapid production of soluble recombinant protein using high-throughput techniques. This method allows the cloning, expression and identification of soluble protein from a given target gene product by a rapid robust method. This ability to produce and analyse soluble recombinant protein in a rapid time period represents a significant advance in an area which has long been considered a significant production bottleneck in the field.

### INTRODUCTION

The recombinant production of protein in bacteria, yeast, insect and mammalian cell lines has become a cornerstone of biological research and the biotechnology industry. Classical biochemical and chromatographical purification techniques usually produce inadequate amounts of a target protein to study its roles or actions. Even if enough of the protein can be purified, it usually involves cumbersome amounts of starting material or tissue and many processing steps are taken before reasonable purification can be achieved.

Recombinant expression of the target protein bypasses a lot of these problems. By introducing the target protein's gene template to a cell line or bacterial culture, induced overexpression can result in significant levels of that protein being produced. Large amounts of protein make the purification a lot simpler, but the addition or fusion of purification domains or tags allows for a relatively simple one-step purification using affinity chromatography resins.

Bacteria, and more specifically, E. coli are ideal expression vehicles for the production of recombinant protein, as large amounts of foreign protein can be expressed in small culture volumes at low cost in comparison with other methods, for example mammalian cell culture. However, the use of bacteria as expression hosts are not without problems. One of the most troublesome shortcomings of the use of E. coli is the production of the recombinant protein in an insoluble form, especially a problem when the target gene is non-bacterial.

Generally, insolubility is the result of the production of protein that is not recognised by the folding enzymes, or chaperones, present in the bacterial cytoplasm. The unfolded or misfolded protein will attempt to decrease its own entropy to a minimum, and it is thought that in an effort to hide or mask its hydrophobic residues from the aqueous environment, the protein molecules aggregate. These aggregates are insoluble and are called inclusion bodies. While in the form of inclusion bodies, the protein will have no biological activity and will be impossible to purify using affinity fusion tags. These inclusion bodies can be re-solubilised in chaotropic buffers such as 8M urea or 6M guanidine hydrochloride, but then must be slowly dialysed against physiological buffers in an effort to refold and regain biological function. Due to the individual characteristics of each protein, this is a slow and painstaking process that may never produce active or useful protein. Therefore, the ability to quickly produce and screen soluble protein in bacteria such as E. coli represents a major step forward in protein biochemistry.

### SUMMARY OF THE INVENTION

The following methodology presented describes a high-throughput process for the cloning, expression and analysis of recombinant soluble protein and protein domains. This process incorporates evaluation and comparison of many factors and conditions known to influence protein solubility at each step in order to guarantee generation of soluble recombinant protein.

According to the present invention there is provided a method of producing a soluble bioactive domain of a protein the method comprising the step of selecting suitable soluble subunits of a protein and assessing the produced protein for desired activity.

The method may comprise the steps of amplifying DNA encoding at least one candidate soluble domain, cloning the amplified DNA into at least one expression vector, using each of said vectors into which the DNA has been cloned to each transfect or transform one or more host cell strains, expressing said DNA in one or more host cell strains, and analysing expression products from said host cells for solubility.

Typically the method comprises the steps of analysis of DNA coding for the protein of interest to identify antigenic soluble domains, designing oligonucleotide primers to amplify DNA encoding the domain, amplifying DNA, cloning the DNA, optionally screening clones for correct orientation of DNA, expressing DNA in expression strains, analysing expression products for solubility, analysing products and production of soluble bioactive protein domain.

The method optionally comprises the step of producing a soluble bioactive protein domain of said protein of interest.

In preferred embodiments of the method according of the invention at least three candidate soluble domains are selected and used in the method in parallel. Thus, in preferred embodiments, each stage of the method of the invention is performed for each domain in parallel i.e. primers are designed for each domain in parallel, prior to amplification and ligation of inserts for each insert being performed in parallel prior to propagation of clones being performed in parallel. However, according to this embodiment, although preferred, it is not essential that each stage of the method is completed for all domains prior to the next stage of the method being initiated for one or more domains. There may be slight staggering of stages of the method between domains by e.g. one or two days.

To further increase the success of the method DNA encoding each selected domain is preferably amplified under at least two, preferably at least three different PCR programs in parallel.

Preferably, in the method of the invention, the amplified DNA encoding each domain is cloned into a plurality of different expression vectors. Such vectors may include any one or more of a vector capable of encoding a fusion protein with a poly-Histidine tag, a vector capable of conferring tight regulation of translation to impose stringent expression conditions, a vector capable of encoding a fusion protein with a solubility enhancing tag. Typically, the solubility enhancing tag is chosen from the group consisting of a glutathione-S-transferase tag, a dihydrofolate reductase tag, a NusA tag and a SNUT tag.

In preferred embodiments, the vectors are each transfected or transformed into a plurality of different host cell strains, preferably different E. coli strains.

As described below, in developing the method of the present invention, the inventors have developed a novel purification tag based on the gene product of a sortase gene, in particular the srtA gene of Staphylococcus aureus. This tag, known as SNUT [Solubility eNhancing Unique Tag] has been found to have exceptional activity, enabling the efficient purification of soluble domains of a number of proteins hitherto not able to be isolated efficiently using conventional purification tags. Throughout this specification, reference to a SNUT Tag should be understood to mean a tag derived from a sortase gene product.

In preferred embodiments, the sortase gene product is a gene product of the srtA gene of Staphylococcus aureus.

Accordingly, in preferred embodiments of the method of the invention, vectors capable of encoding a fusion protein with a SNUT tag are used.

However, utility of the SNUT Tag is not limited to use in the method of the present invention. Indeed in a second independent aspect of the invention, there is provided a purification tag comprising a sortase, e.g srtA, gene product.

Also provided is the use of a sortase, e.g srtA, gene product as a purification tag.

Furthermore, according to a third aspect of the invention, there is provided an expression construct for the production of recombinant polypeptides, which construct comprises an expression cassette consisting of the following elements that are operably linked: a) a promoter; b) the coding region of a DNA encoding a sortase, eg srtA gene product as a purification tag sequence; c) a cloning site for receiving the coding region for the recombinant polypeptide to be produced; and d) transcription termination signals.

According to a fourth aspect of the invention, there is provided a method for producing a polypeptide, comprising: a) preparing an expression vector for the polypeptide to be produced by cloning the coding sequence for the polypeptide into the cloning site of an expression construct according to the third aspect of the invention; b) transforming a suitable host cell with the expression construct thus obtained; and c) culturing the host cell under conditions allowing expression of a fusion polypeptide consisting of the amino acid sequence of the purification tag with the amino acid sequence of the polypeptide to be expressed covalently linked thereto; and, optionally, d) isolating the fusion polypeptide from the host cell or the culture medium by means of binding the fusion polypeptide present therein through the amino acid sequence of the purification tag.

The expression construct, herein referred to as pSNUT, may be made by modification of any suitable vector to include the coding region of a DNA encoding a sortase. In preferred embodiments, the expression construct is based on the pQE30 plasmid.

A sample of pSNUT was deposited with the National Collections of Industrial and Marine Bacteria Ltd. (NCIMB), 23 St Machar Drive, Aberdeen, Scotland AB24 3RY on 23 Dec. 2002 under accession no NCIMB 41153.

In a fifth aspect, there is provided a fusion polypeptide obtained by the method of the fourth aspect of the invention.

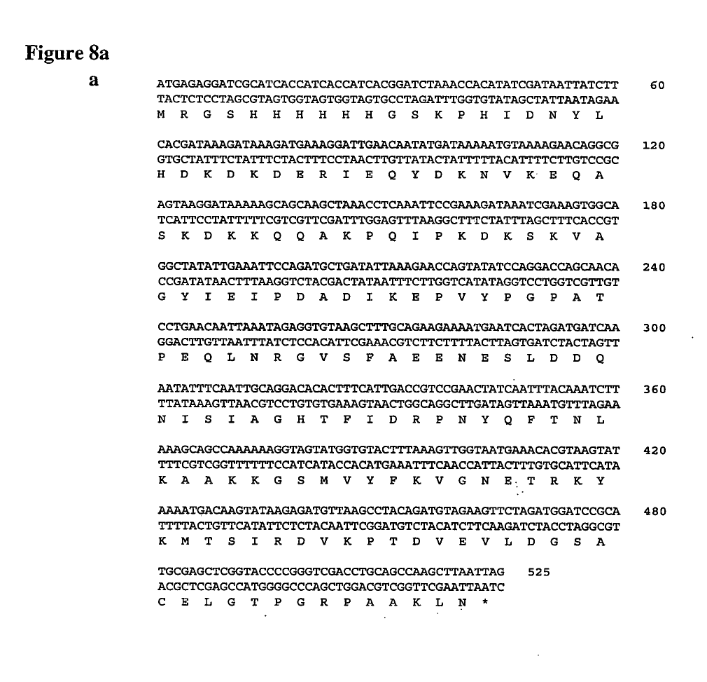

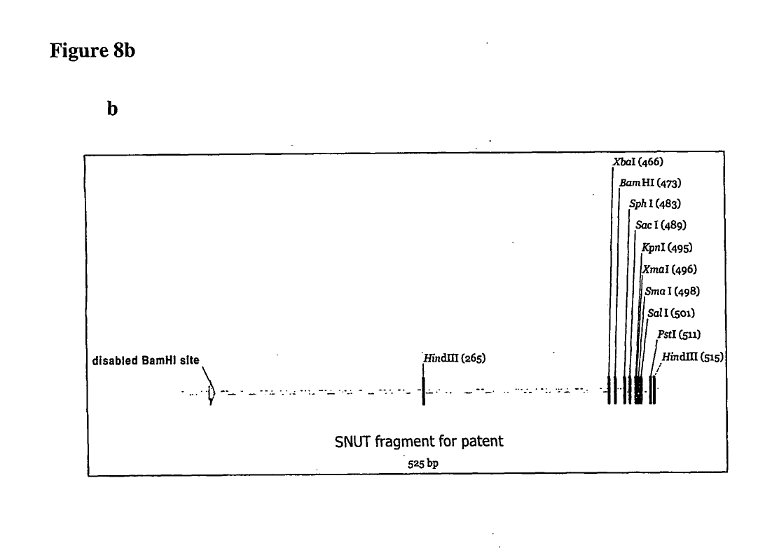

In preferred embodiments, the sortase, e.g. srtA, gene product (SNUT) is encoded by the nucleotide sequence shown in FIG. 8 or a variant or fragment thereof. Preferably, the srtA gene product comprises amino acids 26 to 171 of the SrtA sequence shown in FIG. 8 or a variant or fragment thereof.

Variants and fragments for use in the invention preferably retain the functional capability of the polypeptide i.e. ability to be used as a purification tag. Such variants and fragments which retain the function of the natural polypeptides, can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York.

A variant nucleic acid molecule shares homology with, or is identical to, all or part of the coding sequence discussed above. Generally, variants may encode, or be used to isolate or amplify nucleic acids which encode, polypeptides which are capable of ability to be used as a purification tag.

Preferred variants include one or more of the following changes(using the annotation of AF162687): nucleotide 604 AΔG causing an amino acid mutation of KΔR; nucleotide 647 AΔG, codon remains K, therefore a silent mutation; nucleotide 966 GΔA causing an amino acid mutation of GΔQ.

Variants of the present invention can be artificial nucleic acids (i. e. containing sequences which have not originated naturally) which can be prepared by the skilled person in the light of the present disclosure. Alternatively they may be novel, naturally occurring, nucleic acids, which may be isolatable using the sequences of the present invention. Thus a variant may be a distinctive part or fragment (however produced) corresponding to a portion of the sequence provided in FIG. 8. The fragments may encode particular functional parts of the polypeptide.

The fragments may have utility in probing for, or amplifying, the sequence provided or closely related ones.

Sequence variants which occur naturally may include alleles or other homologues (which may include polymorphisms or mutations at one or more bases). Artificial variants (derivatives) may be prepared by those skilled in the art, for instance by site directed or random mutagenesis, or by direct synthesis. Preferably the variant nucleic acid is generated either directly or indirectly (e. g. via one or amplification or replication steps) from an original nucleic acid having all or part of the sequences of FIG. 8. Preferably it encodes a polypeptide which can be used a s a purification tag.

The term ‘variant’ nucleic acid as used herein encompasses all of these possibilities. When used in the context of polypeptides or proteins it indicates the encoded expression product of the variant nucleic acid.

Homology (i. e. similarity or identity) may be as defined using sequence comparisons are made using FASTA and FASTP (see Pearson & Lipman, 1988. Methods in Enzymology 183: 6398). Parameters are preferably set, using the default matrix, as follows: Gapopen (penalty for the first residue in a gap): −12 for proteins/−16 for DNA Gapext (penalty for additional residues in a gap): −2 for proteins/−4 for DNA KTUP word length: 2 for proteins/6 for DNA. Homology may be at the nucleotide sequence and/or encoded amino acid sequence level. Preferably, the nucleic acid and/or amino acid sequence shares at least about 60%, or 70%, or 80% homology, most preferably at least about 90%, 95%, 96%, 97%, 98% or 99% homology with the sequence shown in FIG. 8.

Thus a variant polypeptide in accordance with the present invention may include within the sequence shown in FIG. 8, a single amino acid or 2, 3, 4, 5, 6, 7, 8, or 9 changes, about 10, 15, 20, 30, 40 or 50 changes. In addition to one or more changes within the amino acid sequence shown, a variant polypeptide may include additional amino acids at the C terminus. and/or N-terminus.

Naturally, regarding nucleic acid variants, changes to the nucleic acid which make no difference to the encoded polypeptide (i. e. ‘degeneratively equivalent’) are included within the scope of the present invention.

Changes to a sequence, to produce a derivative, may be by one or more of addition, insertion, deletion or substitution of one or more nucleotides in the nucleic acid, leading to the addition, insertion, deletion or substitution of one or more amino acids in the encoded polypeptide. Changes may be by way of conservative variation, i. e. substitution of one hydrophobic residue such as isoleucine, valine, leucine or methionine for another, or the substitution of one polar residue for another, such as arginine for lysine, glutamic for aspartic acid, or glutamine for asparagine. As is well known to those skilled in the art, altering the primary structure of a polypeptide by a conservative substitution may not significantly alter the activity of that peptide because the side-chain of the amino acid which is inserted into the sequence may be able to form similar bonds and contacts as the side chain of the amino acid which has been substituted out. This is so even when the substitution is in a region which is critical in determining the peptides conformation.

Also included are variants having non-conservative substitutions. As is well known to those skilled in the art, substitutions to regions of a peptide which are not critical in determining its conformation may not greatly affect its activity because they do not greatly alter the peptide's three dimensional structure.

In regions which are critical in determining the peptides conformation or activity such changes may confer advantageous properties on the polypeptide. Indeed, changes such as those described above may confer slightly advantageous properties on the peptide e. g. altered stability or specificity.

The invention is exemplified with reference to the following non limiting description and the accompanying figures in which

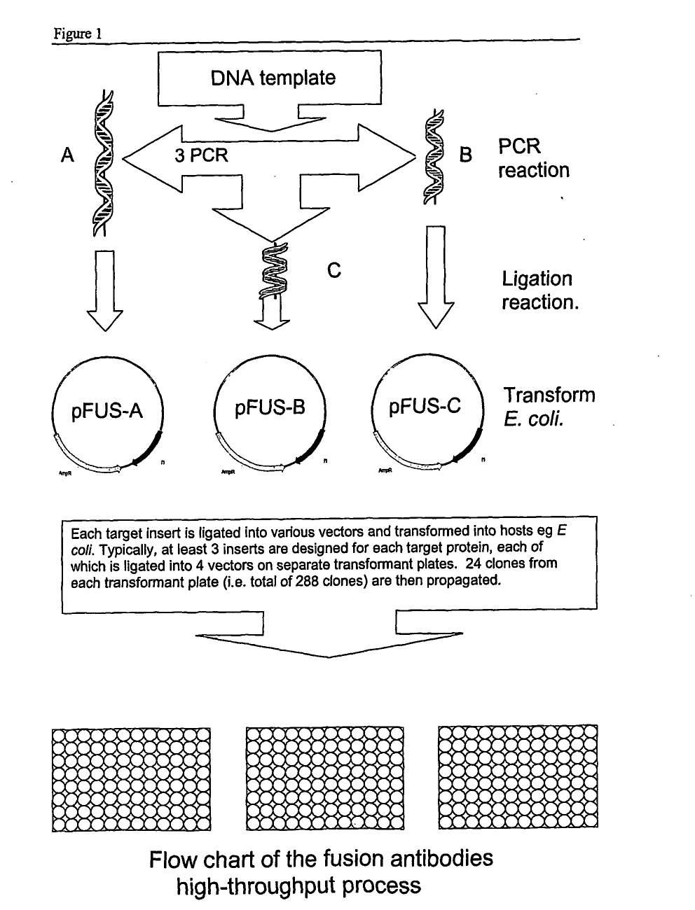

FIG. 1 illustrates the basic protocol used in an embodiment of the invention.

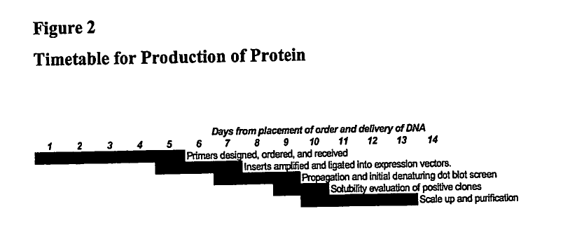

FIG. 2 shows a putative timetable for the process from analysis of the protein to expression of immunisation-ready protein.

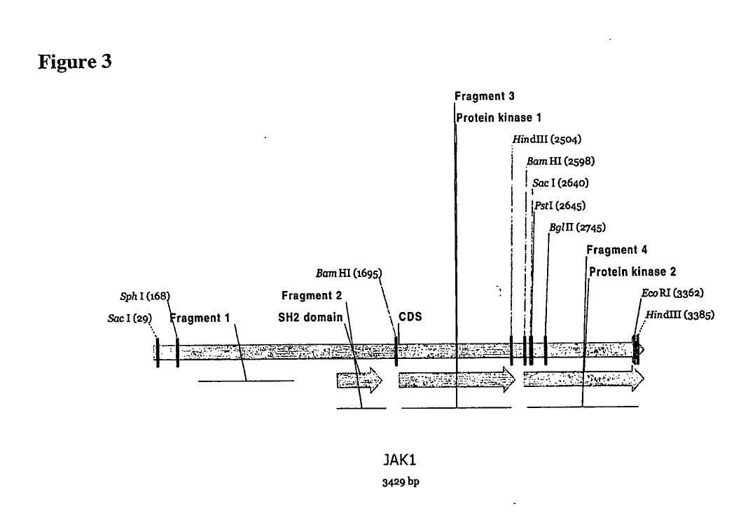

FIG. 3 shows selected domains for amplification from in silico analysis. Representation of a candidate protein for the expression platform, in this case Jak1 (human). Four fragments have been chosen by analysis as depicted.

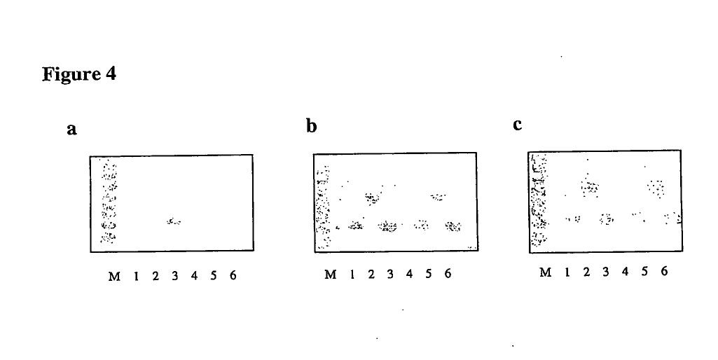

FIG. 4 shows amplification of target domains of the human gene SOCS6 by PCR. Agarose electrophoresis results of the amplification of three fragments from a CDNA clone of the human gene SOCS6. (a) shows domain a (lane 1); domain b (lane 2) and domain c (lane 3) results of amplification using the anticipated annealing temperature as calculated by primer design software as described. Lanes 4-6 show the same amplification procedures using 5% DMSO for inserts a, b and c respectively. (b). Amplification of domains a, b and c using touchdown program in the absence of DMSO (1,2 and 3) and in the presence of 5% DMSO (lanes 4,5 and 6). (c). Amplification of same domains using 50° C. annealing temperature, again in the absence of DMSO (1, 2 and 3), and in the presence of 5% DMSO (lanes 4,5 and 6).

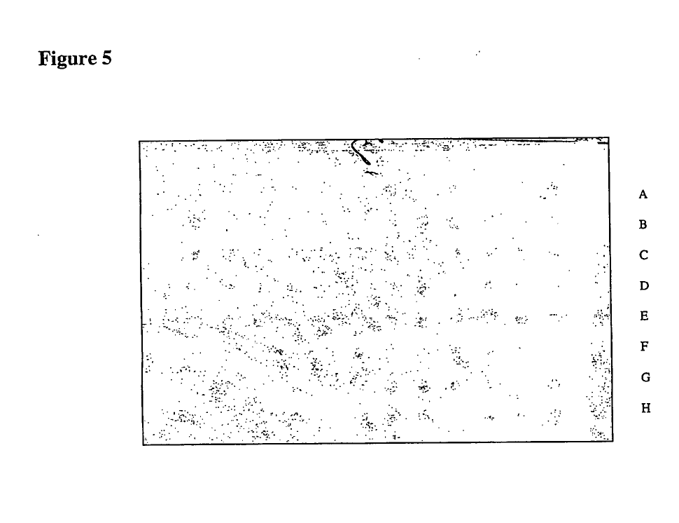

FIG. 5 shows denaturing dot-blot analysis of expression clones of fragments of MAR1 in pQE30.

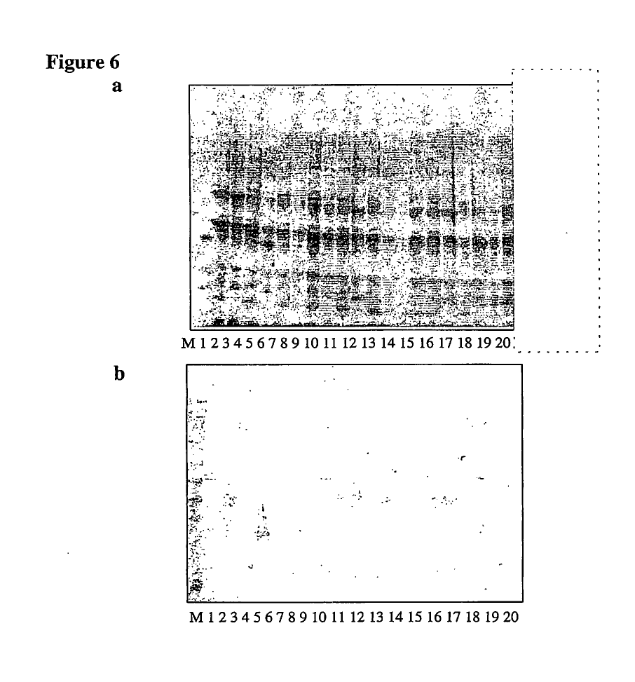

FIG. 6 shows SDS-PAGE and Western blot analysis of soluble lysates. Total protein staining of a 4-20% Bio-Rad Criterium SDS-PAGE gel using chloroform (a), followed by subsequent western blotting of same gel and detection of bands using monoclonal antibody-HRP to poly-histidine tag (b). Results correspond to individual clones expressing NusA-Yotiao protein fusions.

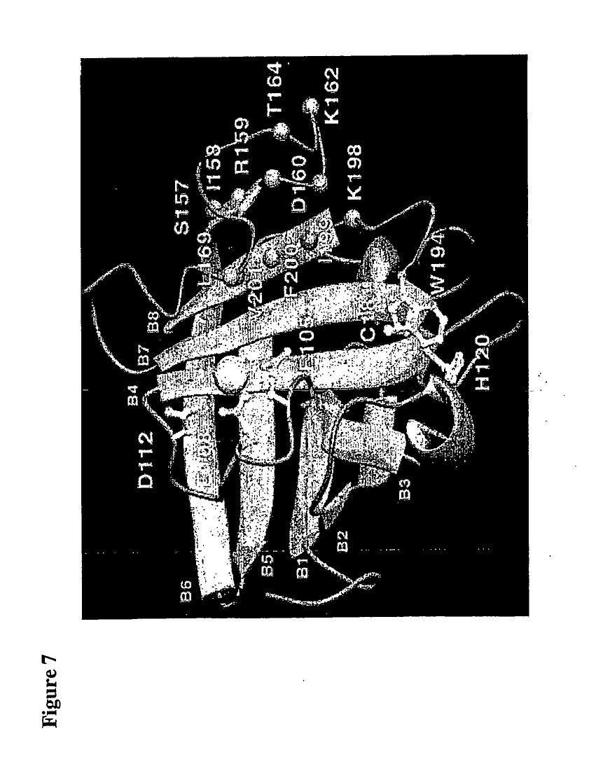

FIG. 7 shows a ribbon Diagram of Staphylcoccus aureus sortase. Ribbon diagram of the putative structure of S. aureus SrtA protein (minus its N-terminal membrane anchor). SNUT represents the portion of this structure between the two yellow arrows as shown. The yellow ball signifies a Ca²⁺ ion, essential for the biological activity of this protein. This diagram is taken from Ilangovan et al., 2001, PNAS 98 (11) 6056 (doi:10.1073/pnas.101064198)

FIG. 8 shows the Nucleotide Sequence and amino acid sequence of SNUT fragment
(a) This is the determined sequence of SNUT. The fragment was cloned into pQE30 using the BamHI site of this vector. When in the wanted orientation, insertion results in the inactivation of the upstream cloning site, therefore allowing any subsequent cloning of target inserts with the downstream BamHI site (see (b) for restriction map of sequence).

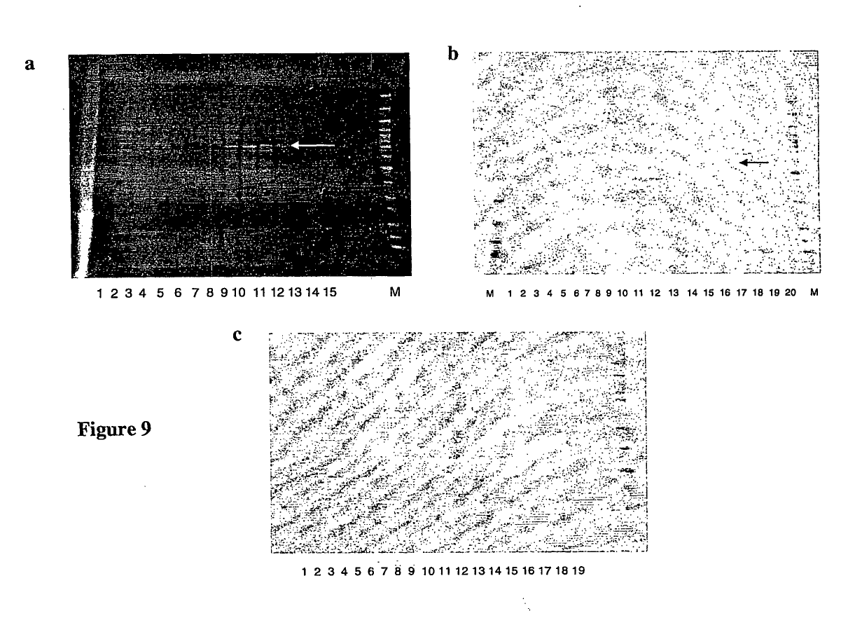

FIG. 9 illustrates qualitative purification results using the SNUT fusion tag. (a) shows the elution profile on SDS-PAGE of SNUT-Jak1 using AKTA Prime native histag purification. Successful elution of SNUT-Jak1 construct is signified by the white arrow. (b) shows the elution profile on SDS-PAGE of SNUT-MAR1 using AKTA Prime native histag purification. Successful elution is shown by the arrow. (c) shows the same gel stained in (b) western blotted and detected using poly-histidine-HRP antibody. This is confirmation that the eluted species in (b) is actually SNUT-MAR1, of expected molecular weight.

### TEMPLATE ANALYSIS AND PRIMER DESIGN

The high throughput process begins with the analysis of the DNA coding for the protein of interest. Software packages such as Vector NTI (Informax, USA) and BLASTP(http://www.ncbi.nlm.nih.gov/BLAST/), p-fam (www.sanger.ac.uk/pfam) and TM pred (www.hgmp.mrc.ac.uk) may be used to identify complete domains within the protein that significantly increase the likelihood of antigenicity and/or solubility when expressed as a subunit of the original protein coding sequence. In order to increase the possibility of identifying a soluble domain, preferably multiple sub-domains, more preferably at least three sub-domains, for example 3 to 9 sub-domains are identified for processing. This has proven optimal to produce soluble protein with the majority of proteins expressed using the method of the invention.

The next step in the process is to design oligonucleotide primers to amplify the selected sub-domains. Primer design may be aided by use of commercially available software packages such as the internet software package Primer3 (http://www-genome.wi.mit.edu/genome software/other/primer3.html) (Whitehead Institute for Biomedical Research), Vector NTI (www.informaxinc.com) and DNASIS (Hitachi Software Engineering Company) (www.oligo.net). These packages allow full control over all aspects of primer design, ranging from primer length, homology to optimal annealing temperature of the PCR reaction itself.

Typically primers for use in the method of the invention are in the range 10-50 base pairs in length, preferably 15 to 30, for example 20 base pairs in length, with annealing temperatures in the range 45-72° C., for example 50-60° C., more conveniently 55-60° C. Primers may be synthesised using standard techniques or may be sourced from commercial suppliers such as Invitrogen Life Technologies (Scotland) or MWG-Biotech AG (Germany).

PCR of Insert

The desired inserts which encode the selected sub-domains are amplified using the primers designed specifically for that target gene using standard PCR techniques. The template DNA for amplification can be in the form of plasmid DNA, cDNA or genomic DNA, depending on whatever is appropriate or indeed available. Any suitable DNA polymerase may be used, for example, Platinum Taq, Pfu (www.stratagene.com) or Pfx (www.invitrogen.com). Any suitable PCR system may be used. In the examples detailed herein, the Expand High Fidelity PCR system (Roche, Basel, Switzerland), was used with working stocks of each primer made (10 pMol/μl).

In preferred embodiments of the invention, several different thermocycler conditions are used with each set of primers. This increases the chance of the PCR working without having to individually optimise each new primer set. Typically the following three programs are used in the method of the invention:
1. A standard PCR programme using the recommended annealing temperature provided with the primers.
2. A standard PCR programme using 50° C. as the temperature for annealing.
3. A touchdown PCR programme, where the annealing temperature starts at a high temperature e.g 65° C. for 10 cycles and then gradually decreases the annealing temperature to 50° C. over the subsequent e.g 15 cycles.

Buffer conditions may be adjusted as required, for example with respect to magnesium ion concentration or addition of DMSO for the amplification of difficult templates.

The PCR products are then visualised using standard techniques, for example on a 1.5% agarose gel stained with Ethidium Bromide and the bands are cut out of the gel and purified using Mini elute gel extraction Kit (Qiagen, Crawley, England).

Expression Vectors

Amplified DNA inserts are subsequently cloned into expression vectors using techniques dictated by the multiple cloning sites of the vector in question. Such techniques are readily available to the skilled person.

In order to maximise the successful generation of soluble antigen, the amplified DNA coding for each target protein domain is preferably cloned into a plurality of different expression vectors. This allows the generation of a library of novel expression constructs which can then simultaneously be screened for the high level production of soluble protein. Each construct will have different properties due to attachment of ‘tag’ domains, which are designed to increase expression and solubility.

Any suitable expression system can be used in the method of the invention. Preferably, the expression system is prokaryotic. Preferably at least two expression vectors, preferably three, most preferably 4 to 5 vectors are used for each of the constructs in the method of the invention. Preferably, vector combinations are chosen to allow the same cloning methodologies to be used simultaneously as this allows a much more rapid entry in expression trials.

Suitable vectors for use in the method of the invention include one or more of the following:
I. Vectors that will generate fusion protein with a poly-Histidine tag (his-tag, hexahistidine tag, or his-patch). The expressed His tag can be situated at either the N or C terminus of the protein, or even internally. Examples include the pQE series from Qiagen, Valencia, Calif.; pET 14-19, Novagen, Madison, Wis. A poly-histidine tag is an non-natural amino acid sequence with unusual and specific chelation properties with metal bivalent ions such as Ni²⁺ and Cu²⁺. Immobilised metal affinity chromatography (IMAC) exploits this property to allow the specific purification of proteins containing this tag, therefore making it an extremely useful purification tool.
II. Vectors that confer tight regulation of translation to impose stringent expression conditions especially for proteins that are toxic to a prokaryotic host. An example of such a vector is the pQE80 vector, Qiagen. Tight regulation is absolutely essential for the production of some proteins, especially proteins foreign to the bacterial host which are more likely to have toxic effects to the bacterial host. Some high-level expression systems are not particularly stringent and leaky expression may occur without induction, causing bacterial hosts to be killed before a culture has reached a great enough density to sustain expression of a toxic gene.
III. Vectors that will generate fusion proteins with a solubility enhancing tag such as glutathione-S-transferase (examples include the pGEX series, Amersham Biosciences, Uppsala, Sweden; pET41/2, Novagen) or NusA (pET43, Novagen). These tags have been identified as proteins of a highly soluble nature in E. coli and confer their soluble characteristics to proteins attached to them as fusion partners.
IV. Vectors that encode fusion partners that facilitate the expression of small or poorly expressed proteins including glutathione-S-transferase and dihydrofolate reductase (Amersham Biosciences and Qiagen respectively). Some proteins, due to the composition of the coding DNA are only poorly expressed in bacteria. In some cases they may not be produced at all. Tags such as GST and DHFR can aid such expression if incorporated as N-terminal fusions to help generate adequate amounts of a target protein, where no protein would be expressed if the template was only the target DNA.
V. Vectors that encode SNUT. [Solubility eNhancing Unique Tag], for example pSNUT. This tag is based on the sequence of a trans-peptidase found on the surface of gram-positive bacteria. This protein is highly soluble, and expressed as very high levels. As described below, the inventors have found that SNUT is an ideal fusion tag for conferring solubility and expression levels to target protein fragments. SNUT may be cloned into any suitable vector. For the purposes of the results shown in this application, the sequence incorporating the SNUT fragment is cloned into pQE30 in a manner allowing full use of the multiple cloning site (MCS) of this vector for downstream gene insertions.
   Development of pSNUT

Occasionally, due to the varying nature of proteins, the production of soluble protein has remained elusive. In fact in some cases, production of protein can be a problem due to differences in the machinery of bacterial cells. During the development of this high-throughput expression platform, the need for a more versatile tag than is available currently on the market became evident.

The inventors found that a tag based on the srtA gene product from Staphylcoccus aureus is highly soluble nature, reacts well to purification schemes and expresses particularly well. It was hypothesised that the incorporation of a portion or domain of this protein could represent a useful fusion tag in the present method, and indeed the expression of any poorly soluble protein in E. coli. Using NMR studies, the 3D structure of this protein has been predicted and is shown in FIG. 7. We hypothesised that by taking a portion of this structure, we could make a manipulatable protein tag, but not disturb its tertiary structure enough to reduce its highly favourable characteristics listed above. The region of this protein used as a solubility-enhancing tag is depicted by two arrows.

To make this tag compatible with the other vectors and systems being used on the platform, this SNUT tag was cloned into pQE30 as described earlier. However, it may be cloned into any suitable expression vector. Positive clones may be identified by denaturing dot blots, SDS-PAGE and Western blotting. Final confirmation of these clones was provided by DNA sequencing, and the sequence of the multiple cloning region of the resultant vector is shown in FIG. 8.

Variances in the sequence of the SNUT domain were observed from the sequence for SrtA that has been logged in Genbank (AF162687). The variances are (using the annotation of AF162687) nucleotide 604 AΔG causing an amino acid mutation of KΔR; nucleotide 647 AΔG, codon remains K, therefore a silent mutation; nucleotide 966 GΔA causing an amino acid mutation of GΔQ.

Preliminary trials and native purification showed that the SNUT fragment was very soluble and its characteristics were in no way diminished by truncation, thus showing that SNUT could represent a useful tag domain (data not shown). As described in the Examples, to fully test the abilities of SNUT, we then chose two proteins were soluble protein production had proved impossible using conventional methods and using the other expression systems of the method of the present invention. Surprisingly, we found that, using pSNUT in the method of the invention, these proteins could be produced in soluble form.

Accordingly, in preferred embodiments of the method of the invention, at least one of the vectors encodes SNUT.

Clone Propagation

Target insert/expression vector ligations are propagated using standard transformation techniques including the use of chemically competent cells or electro-competent cells. The choice of the host cell and strain for transformation is dependent on the characteristics of the expression vectors being utilised.

In the method of the invention, bacterial cells, for example, Escherchia coli, are the preferred host cells. However, any suitable host cell may be used. In preferred embodiments, the host cells are Escherchia coli.

In preferred embodiments of the present invention, in order to further maximise the chances of success in isolating a soluble protein, one or more, preferably all of the vectors are used to each transfect or transform a plurality of different host cell strains. The set of host cell strains for individual vector may be the same or different from the set used with other vectors.

In a particularly preferred embodiment of the invention, each vector is transformed into three E. coli strains (for example, selected from Rosetta(DE3)pLacI, Tuner(DE3)pLacI, Origami BL21 (DE3)pLacI and TOP10F, Qiagen).

Where the vectors are pQE based vectors, TOP10F′ cells are preferred for the propagation and expression trials of such vectors. The present inventors have identified this strain as a more superior strain for these vectors than either of the recommended strains by the supplier (M15(pREP4) and SG13009(pREP4)), in terms of ease of use and culture. maintenance (only one antibiotic required as to two with M15(pREP4) or SG13009(pREP4) (www.quiagen.com). Other F′ strains such as XL1 Blue can be used, but are inferior to the TOP10F′ strain, due to lack of expression regulation (results not shown). The use of TOP10F′ (Invitrogen) for the propagation and/or expression pQE based vectors forms an independent aspect of the present invention. Other F′ strains such as XL1 Blue may also be used, but are inferior to the TOP10F′.

After transformation, cells are plated out onto selection plates and propagated for the development of single colonies using standard conditions.

Propagation of Cells

In preferred embodiments, the colonies are used to inoculate wells in a 96 well plate.

Routinely, 6-48 clones for each insert-vector ligation are taken and propagated in culture micro-titre plates containing up to 500 μl of media.

Typically, each well may contain 200 μl of LB broth with the appropriate antibiotics. Each plate is dedicated to one strain of E. coli or other host cell which alleviates the problems of different growth rates. The necessary controls are also included on each plate. The plates are then grown up, preferably at 37° C. or any other temperature as appropriate to the particular host cell and vector, with shaking, until stationary phase is reached. This is the primary plate.

From the primary plate a secondary plate is seeded and then grown to log phase. Typically, the secondary plate is seeded using ‘hedgehog’ replicators. Determination of positive clones from these plates may be undertaken using functional studies. According to the conditions and reagents required, protein production is then induced, and cultures propagated further. Most vectors are under the control of a promoter such as T7, T71ac or T5, and can be easily induced with IPTG during log phase growth. Typically, cultures are propagated in a peptone-based media such as LB or 2YT supplemented with the relevant antibiotic selection marker. These cultures are grown at temperatures ranging from 4-40° C., but more frequently in the range of 20-37° C. depending on the nature of the expressed protein, with or without shaking and induced when appropriate with the inducing agent (usually log or early stationary phase). After induction, growth propagation can be continued for 1-16 hours for a detectable amount of protein to be produced.

The primary plate is preferably stored at 4° C. as a reference, until the process is complete.

Colony Screening for Inserts in Correct Orientation

The method of the invention may include the step of testing transformants for correct orientation of the inserts.

Although all colony selecting and picking can be done manually, automated colony pickers are preferred. Automated colony pickers such as the BioRobotics BioPick allow for the uniform and reproducible selection of clones from transformation plates. Clone selection determinants can be set to ensure picking colonies of a standardised size and shape. After picking and plate inoculation, propagation of clones can be carried out as described above.

Identification of positive clones can be achieved through a variety of methods, including standard techniques such as digestion analysis of plasmid DNA; colony PCR and DNA sequencing. Alternatively, in a preferred embodiment, the novel method of dot-blotting described herein for the identification of positive clones may be used in place of such traditional techniques, prior to final confirmation by DNA sequencing. The use of this method in the platform presented here is not essential in the use of this platform over existing screening methodologies, but represents a rapid, reproducible and robust detection method. The protocol described here is a new protocol for an existing method for which commercially available equipment (Bio-Rad DotBlot) can be purchased.

This particular method is useful for the rapid detection or presence of recombinant protein and allows for a determination of all clones irrespective of solubility and conformation. This is useful at this stage, because conformational structures can inhibit the detection of tag domains if they are not presented properly on the surface of the protein. This can occur as easily with both soluble and insoluble protein.

For example, after growth on the micro-titre plates is complete, the plate is centrifuged at 4000 rpm for 10 minutes at 4° C. to harvest the bacterial cells. The supernatant is removed and the cell pellets are re-suspended in 50 μl lysis buffer (10 mM Tris.HCl, pH 9.0, 1 mM EDTA, 6 mM MgCl₂) containing benzonase (1 μl/ml). The plate is subsequently incubated at 4° C. with shaking for 30 minutes. A sample (10 μl) of the cell lysate is added to 100 μl buffer (8 M urea, 500 mM NaCl, 20 mM sodium phosphate, pH 8.0) and incubated at room temperature for 20 minutes. Samples are then applied to a BioDot apparatus (BioRad) containing nitrocellulose membrane (0.45 μM pore size) in accordance with the manufacturers' instructions. The membrane is removed and transferred into blocking reagent (3% w/v; Bovine serum albumin in TBS) for 30 minutes at room temperature. The blot is washed briefly with TBS then incubated in a primary antibody, specific to the tag being used for the subset of expression clones. Depending on the nature of the primary i.e., whether or not it has a horse radish peroxidase (HRP) reporter function, will depend on whether the use of a secondary is required. For detection of specific binding the membrane is then washed 2×5 minutes in TBS followed by 1×5 minute wash in 10 mM Tris.HCl pH 7.6. Detection of specifically bound antibody is disclosed by the addition of chromogenic substrate (6 mg diaminobenzidine in 10 ml 10 mM Tris.HCl pH 7.6 containing 50 μl 6% H₂O₂). The reaction is stopped by thorough rinsing in water. Positive clones identified by this procedure can then be confirmed by DNA sequencing of the expression construct using now industry-standard techniques and equipment such as ABI and Amersham Biosciences.

Sequencing

The sequencing reactions may be performed using techniques common in the art using any suitable apparatus. For example, sequencing may be performed on the cloned inserts, using the Big Dye Terminator cycle sequencing kits (Applied Biosystems, Warrington, UK) and the specific sequencing primer run on a Peltier Thermal cycler model PTC225 (MJ Research Cambridge, Mass.). The reactions may be run on Applied Biosystems—Hitachi 3310 Sequencer according to the manufacturer's instructions. These sequences are checked to ensure that no PCR generated errors have occurred.

Assessment of Solubility of Positive Clones

The cells of the positive clones may then be harvested and soluble and insoluble protein detected.

Any suitable techniques known in the art can be used to separate soluble and insoluble protein, such as the use of centrifugation, magnetic bead technologies and vacuum manifold filtrations. Typically, however, the separated proteins are ultimately analysed by acrylamide gel and western blotting. This confirms the presence of recombinant protein at the correct size.

In one embodiment, contents of each well in the 96 well plate are transferred into a Millipore 0.65 μm multi-screen plate. The plate is placed on a vacuum manifold and a vacuum is applied. This draws off the culture medium to waste. The cells are then washed with PBS (optional), again the vacuum is applied to remove the PBS. The multi-screen plate is removed from the manifold and bacterial cell lysis buffer (containing DNAse) (50 μl) is added to each well. The plate is incubated at room temperature for 30 minutes with shaking to facilitate lysis of the cells. A fresh 96 well microtitre plate is placed inside the vacuum manifold and the multi-screen plate is placed above it. When a vacuum is applied the contents of each well are drawn into the micro-titre plate below. The vacuum only needs to be applied for 20 seconds. The collected lysate contains the soluble fraction of expressed protein. A sample of the collected lysate may subsequently analysed by SDS-PAGE and Western blotting to confirm both the presence and correct molecular weight of the target protein.

The use of SDS-PAGE and Western blotting can be expensive and time consuming, especially when numerous samples must be analysed for each construct. In light of this we have developed a protocol whereby one gel can be used for both total protein staining and western blotting. This represents a significant improvement in this methodology and obviously allows cost saving, and precise comparisons can be made with regard to total protein and western blotting as both sets of results come from the one gel.

The basis of this protocol is in the ability to use chloroform and UV light to stain protein on an SDS-PAGE gel (Kazmin et al., Anal Biochem, 2002, 301(1) 91-6; doi:10.1006/abio.2001.5488). We have used this technique to great effect as it allows for the extremely rapid staining of a SDS-PAGE gel in less than a tenth of the time taken using other more traditional staining methods such as Commassie Brilliant Blue and Collodial Blue stains. We then decided to take this observation a step further and analyse the ability of a chloroform-stained gel to be used in Western blotting. This would not be expected to work as other stained gels result in the fixing of the protein to the gel and subsequent inability to transfer the protein during blotting. This expectation is coupled to the fact that chloroform is not compatible with western blotting equipment (Bio-Rad SD blotter user's manual). However, fortuitously, we have discovered that with a wash of the chloroform-stained gel in double-distilled water, to remove excess chloroform, and after subsequent soaking in transfer buffer, proteins were effectively transferred during western blotting in contrast to expectations. This transfer was no-less effective than from a gel that has not been pre-stained with chloroform and UV light. FIG. 6 primarily shows results relating to the production of soluble protein by the platform, but also shows the ability to use the chloroform-stained SDS-PAGE derived western blot for the identification of proteins, without any apparent damage caused to the proteins.

Th use of a chloroform-stained SDS-PAGE derived western blot for the identification of proteins forms another aspect of the present invention.

Scale-Up and Purification

This analysis provides a picture of the expression status of the clones on each plate. Using this analysis, positive soluble protein expressing clones can be identified for the production of soluble recombinant protein for a given target protein. The clones may be selected and their growth scaled up e.g. to 5 ml scale, using the saved primary plate as an inoculum. Parameters that may be taken into consideration in deciding on the appropriate culture to select for scale-up include the desirability of specific regions for the production of an antigen, the overall expression levels of the clone and factors that may affect affinity purification such as amino acid composition.

### EXAMPLE 1

Overview of Process

FIG. 1 illustrates the basic protocol used in an embodiment of the invention. The DNA coding for the protein of interest is analysed to identify target domains which may enhance solubility. For each insert, multiple primers are designed and used to amplify the Chosen nucleotide sequences. For each primer set, the PCR reaction is performed under three different thermocycler conditions: a standard PCR programme using the recommended annealing temperature provided with the primers; a standard PCR programme using 50° C. as the temperature for annealing; and a touchdown PCR programme, where the annealing temperature starts at 65° C. for 10 cycles and then gradually decreases the annealing temperature to 50° C. over the subsequent 15 cycles.

### EXMAPLE 2

Expression Construct Design

FIG. 3 is a diagrammatic representation of the protein Jak1. Using pfam, the position of distinct domains was established. Further analysis of these domains was then carried out using Tmpred and the Kyle and Dolittle hydrophobicity algorithm to determine the usefulness of these domains as soluble antigens. From this tentative analysis, four domains were selected for amplification and expression analysis.

### EXAMPLE 3

Parallel Amplification of DNA Sequences Under Different PCR Conditions Enables Rapid Amplification of Inserts of Interest

Based on preliminary in silico analysis, primers specific for a target protein were designed and used to amplify domains selected for analysis. FIG. 4 shows the amplification of portions of human SOCS6 gene from a cDNA plasmid clone using three programs:
1. A standard PCR programme using the recommended annealing temperature provided with the primers.
2. A standard PCR programme using 50° C. as the temperature for annealing.
3. A touchdown PCR programme, where the annealing temperature starts at a high temperature e.g 65° C. for 10 cycles and then gradually decreases the annealing temperature to 50° C. over the subsequent e.g 15 cycles.
a) shows domain a (lane 1); domain b (lane 2) and domain c (lane 3) results of amplification using the anticipated annealing temperature as calculated by primer design software. Lanes 4-6 show the same amplification procedures using 5% DMSO for inserts a, b and c respectively. (b). Amplification of domains a, b and c using touchdown program in the absence of DMSO (1,2 and 3) and in the presence of 5% DMSO (lanes 4,5 and 6). (c). Amplification of same domains using 50° C. annealing temperature, again in the absence of DMSO (1, 2 and 3), and in the presence of 5% DMSO (lanes 4,5 and 6). It is clear from these results how much more effective the use of varying protocols (4b and 4c) is over the basic protocol using the pre-determined annealing temperatures. These results show the requirement of different programs to guarantee the amplification of certain inserts, even with gene specific DNA primers, as no strict rules can be applied for the amplification of DNA for every different gene target.

Furthermore, the manipulation of the Mg²⁺ and DMSO in the reaction buffer may be useful for the guaranteed amplification of some gene fragments, as seen in FIG. 4. In the present example, no amplification of a cancer antigen DNA was successful without the addition of DMSO, which was added in order to disrupt secondary structure and cause some denaturing. This allows primers to anneal to some difficult templates prior to elongation by the DNA polymerise during PCR.

These results depict the high-throughput nature of the method of the invention, even at a DNA level. These procedures allow the rapid amplification of all gene inserts

### EXMAPLE 4

Dot Blotting

The optional use of dot-blotting in the method of the invention has proven to be an invaluable tool for the preliminary evaluation of clones for protein expression. FIG. 5 shows the results of a denaturing dot-blot analysis of expression clones of fragments of murine antigen receptor MAR1 in pQE30. using the method of the invention. The blot depicts the expression of all 4 target fragments designed in pQE30, and clearly shows the levels of poly-histidine tagged protein in each well. All detection was achieved using horse radish peroxidase conjugate to a poly-histidine tag monoclonal antibody (Sigma). Rows A and B are 24 individual clones of insert 1 in pQE30. Rows C and D represent insert 2; rows E and F represent insert 3 and G and H represent insert 4. Presence of purple product on an individual dot signifies positive detection of the presence of poly-histidine tag and therefore a positive clone.

### EXAMPLE 5

Evaluation of Soluble Protein From Yotiao.

In this example, results are shown for the expression and analysis of the mammalian gene yotiao. Gene specific primers were designed and used for the amplification of the target regions and these were then cloned into pQE30, pQE80, pGEX and pET43.1a using the following protocol.

Vectors (500 ng) were restricted with BamHI (20 units) and SalI (20 units) in the presence of calf intestinal alkaline phosphatase (CIP) (2 units), gel purified and quantified using standard methods. Purified PCR fragments (100 ng) were restricted with BamHI (5 units) and SalI 5 units), gel purified, quantified, and then used in a ligation reaction with the restricted vector again using standard T4 DNA ligase methods (Ready-to-Go T4 DNA ligase, Amersham Biosciences). A sample of the ligation reaction (1 μl) was then used to transform the appropriate competent bacterial cells (TOP10F′ were used here for the pQE vectors, a modification of the manufacturers recommendations; BL21(DE3)pLysE for pET43.1a and TOP10F′ for pGEX-Fus). Transformants were selected on LB/ampicillin (100 μg/ml) for the pQE and pGEX-Fus vectors and LB/ampicillin/chloriphenicol/glucose for pET43.1 (50 μg/ml, 32 μg/ml and 1% respectively) overnight at 28° C.

A Cambridge BioRobitics BioPick instrument was used for the picking of 24 colonies from each of the transformant plates into flat-bottomed and lidded micro-titre plates. For this screen there were 3 inserts in 4 vectors, resulting in a total of 288 clones picked. All pQE30, 80 and pGEX-Fus clones were used to inoculate 150 μl of LB (containing 100 μg/ml ampicillin) (see FIG. 1), and these were allowed to grow overnight at 37° C. For the pET43.1a clones, LB containing 1% glucose, 50 μg/ml ampicillin and 34 μg/ml chloramphenicol were used for propagation. These pET43.1a clones were grown overnight at 28° C. From this plate, secondary plates were seeded using ‘hedgehog’ replicators, and these are again grown up to log phase prior to induction with IPTG and being left to grow overnight.

A secondary plate was then prepared by the inoculation of 200 μl of LB containing the required supplements with 10 μl of the overnight primary culture. These were then grown at 37° C. (for the pQE30, 80 and pGEX-Fus constructs) and 28° C. (for the pET43.1a clones). Once an optical density (OD) of 0.25 at A550 was reached, IPTG (final concentration, 1 mM) is added to induce expression of the recombinant protein. Culture propagation was continued for another 4 hours prior to harvesting of bacterial cells.

After clones expressing specific recombinant protein have been identified, the solubility of these proteins has to be established prior to clone selection for purification. This can be performed a number of ways including the use of centrifugation and automation-friendly vacuum manifold separations.

The results shown here were obtained using methodologies based around the use of vacuum-assisted filtration to separate soluble and insoluble protein. The filtrates that were produced from the method described were then analysed by SDS-PAGE and Western blotting to confirm the production of a recombinant protein of the correct anticipated molecular weight.

FIG. 6 shows the examination of screened-clone soluble extracts by SDS-PAGE and western blotting. These particular results are for the expressed products of the bacterial gene yotiao from the pET43.1a vector (producing Yotiao fragments as NusA fusion proteins). The SDS-PAGE gel shows the clear presence of expressed soluble protein in the lysates, which is confirmed to contain poly-histidine tags on the accompanying western blot. The results in FIG. 6 are proof of the effectiveness of the method presented here. The production of soluble protein using one of the expression systems, pET43.1a is clearly visible, thus allowing identification of clones suitable for scale-up cultures and subsequent purification. The production of soluble Yotiao protein fragments from the other systems was tried (pQE30; pQE40 and pQE80), but proved unsuccessful. Clones expressing soluble Yotiao were identified and then confirmed by DNA sequencing within 3 weeks of receiving the cDNA template for the gene.

These results collectively show the power and utility of the platform. Normally, expression of such a protein would be carried out in just a basic vector such as pQE30 alone, and inability to produce soluble protein using this system, which is also part of the platform, exemplifies the power of the platform to guarantee soluble recombinant protein production.

### EXAMPLE 7

Design and Construction of SNUT Expression Tag

Based on analysis of the amino acid sequence and predicted structure of SrtA_{ΔN}, it was decided to amplify the region of amino acids 26 to 171 of the SrtA sequence. Amplification was conducted using the forward primer 5′ TTTTTTAGATCTAAACCACATATCGAT and the reverse primer 5′ TTTTTTGGATCCATCTAGAACTTCTAC. This product was then digested with BglI and BamHI and ligated into pQE30 vector which had also been digested with BamHI to form the pSNUT vector. The ligation mix was transformed into TOP10F′ cells and single colonies propagated on LB agar containing 100 μg/ml ampicillin. Clones with the srtA fragment in the correct orientation were screened by expression analysis and positive clones identified using the denaturing dot-blot assay described earlier.

The sequence encoding the SNUT tag was cloned into pQE30 as described earlier and positive clones identified by denaturing dot blots, SDS-PAGE and Western blotting. Final confirmation of these clones was provided by DNA sequencing, and the sequence of the multiple cloning region of the resultant vector is shown in FIG. 8. Variances in the sequence of the SNUT domain were observed from the sequence for SrtA that has been logged in Genbank (AF162687). The variances are (using the annotation of AF162687) nucleotide 604 AΔG causing an amino acid mutation of KΔR; nucleotide 647 AΔG, codon remains K, therefore a silent mutation; nucleotide 966 GΔA causing an amino acid mutation of GΔQ.

### EXAMPLE 8

Trials of SNUT Expression Constructs

Target inserts were cloned into the pSNUT vector using primer construction and digestion of resulting PCR amplifications with BamHI and SalI as described earlier. pSNUT was digested with BamHI in a similar manner and the target inserts cloned as described. Clones were screened using the denaturing dot-blot system and then analysed with SDS-PAGE and western blotting. Positive clones were used for preparative 200 ml LB cultures containing 100 μg/ml ampicillin and induced as described earlier. This was grown to an optical density of 0.5 at A₅₅₀ at 37° C. Expression of SNUT was then induced with the addition of IPTG (final concentration, 1 mM) and left to grow for another 4 hours. Cells were then harvested by centrifugation at 5K rpm for 15 minutes. Cells were re-suspended in 30 ml PBS containing 0.1% Igepal and lysis induced by two freeze-thaw cycles. The suspension was then sonicated and centrifuged at 5K rpm for 15 minutes. The soluble supernatant was transferred to a fresh container and filtered through a 0.8 μm disc filter to remove final cell debris. This solution was then applied to a Ni²⁺ charged IMAC column (Amersham Biosciences HiTrap Chelating column, 1 ml) using an AKTA Prime low pressure chromatography system and column was then treated using a standard native his-tag purification protocol involving washing of column with 20 mM sodium dihydrogen phosphate pH 8.0 containing 10 mM imidazole, 500 mM NaCl, and elution of soluble his-tagged proteins using 20 mM sodium dihydrogen phosphate pH 8.0 containing 500 mM imidazole, 500 mM NaCl. Elution fractions were then analysed on an SDS-PAGE gel (4-20% SDS-PAGE Bio-Rad Criterion gel), which was stained with chloroform as described earlier. This gel was then subsequently western blotted and the his-tagged protein detected with anti-poly-histidine monoclonal antibody as described earlier.

Preliminary trials and native purification showed that the SNUT fragment was very soluble and its characteristics were in no way diminished by truncation, thus showing that SNUT could represent a useful tag domain (data not shown). To fully test the abilities of SNUT, we then chose two proteins for which soluble protein production had proved impossible using the other expression systems in which SNUT was not used as a tag. These were murine MAR1 and human Jak1. Clones were prepared and selected using the method as described in the Examples above and positive clones were subsequently grown and induced at 37° C. These were then treated to identical native histag purifications. Both proteins behaved very favourably under standard purification conditions as can be seen from the purification profiles in FIG. 9. For both these trial proteins, this was the first example of such purification under soluble conditions. The production of these proteins using conventional techniques has failed to produce any soluble protein, irrespective of expression system or growth conditions used (data not shown). However, as described in this example, when the protein fragments were expressed in pSNUT, soluble proteins can be surprisingly obtained.

The effectiveness of SNUT as a fusion protein is even more significant when it is considered that no special growth conditions were required for the generation of soluble protein. This is remarkable when one considers the protein expressionist's standard GST tag which is not even soluble itself when expressed at 37° C.; 28° C. is required before even the generation of GST on its own without any target protein is observed.

In this application we have demonstrated that our high throughput cloning and expression platform can rapidly identify clones that express soluble protein. This is achieved through the use of a number of expression vectors coupled with a range of target fragments. That coupled with our expression conditions; sample processing and analysis ensure that soluble antigen is generated. As can be seen from the results presented, the production of a soluble mammalian protein in E. coli can be troublesome and requires the application of several different methodologies, or expression systems and conditions in order to guarantee a successful outcome. The protocols detailed in this spcification are the ideal automation-ready platform for generation of such soluble protein. This platform offers not only the generation of soluble protein, but also in a rapid, reproducible and robust manner.

All documents referred to in this specification are herein incorporated by reference. Various modifications and variations to the described embodiments of the inventions will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes of carrying out the invention which are obvious to those skilled in the art are intended to be covered by the present invention.

1. A method of producing a soluble bioactive domain of a protein of interest, the method comprising the step of selecting at least one candidate soluble domain of the protein and assessing the produced protein of each domain for desired activity.

2. The method according to claim 1 comprising the step of amplifying DNA encoding at least one candidate soluble domain, cloning the amplified DNA encoding each candidate domain into at least one expression vector, using each of said vectors into which the DNA has been cloned to each transfect or transform one or more host cell strains, expressing said DNA in or more of said host cell strains, and analysing expression products from said host cells for solubility.

3. The method according to claim 2 comprising steps:
(a) analysing DNA coding for a protein of interest to identify one or more candidate soluble domains,
(b) providing oligonucleotide primers to amplify DNA encoding each domain,
(c) amplifying said DNA with said primers,
(d) cloning amplified DNA from step (c) for each domain into at least one expression vector,
(e) optionally screening clones for correct orientation of DNA,
(f) using each of the vectors of step (d) into which the DNA has been cloned to each transfect or transform one or more host cell strains,
(g) expressing said DNA in one or more of said host cell strains, and
(h) analysing expression products from said host cells for solubility.

4. The method according to claim 2 or comprising the step of producing a soluble bioactive protein domain of said protein of interest.

5. The method according to claim 2 wherein at least three candidate soluble domains are selected and DNA is amplified for each of said domains.

6. The method according to claim 2 wherein said DNA encoding each selected domain is amplified under at least two, preferably at least three different PCR programs in parallel.

7. The method according to claim 6 wherein said PCR programs are selected from (i) a standard PCR programme using a predicted annealing temperature for the primers, (ii) a standard PCR programme using a temperature in the range 48 to 52° C., preferably 50° C. as the temperature for annealing and (iii) a touchdown PCR programme, where the annealing temperature starts at a temperature in the range 62 to 67° C., preferably 65° C., and then gradually decreases to a temperature in the range 48 to 52° C., preferably 50° C., over the subsequent cycles.

8. The method according to claim 2 wherein the amplified DNA encoding each domain is cloned into a plurality of different expression vectors.

9. The method according to claim 8 wherein the plurality of vectors include one or more of a vector capable of encoding a fusion protein with a poly-Histidine tag, a vector capable of conferring tight regulation of translation to impose stringent expression conditions, a vector capable of encoding a fusion protein with a solubility enhancing tag.

10. The method according to claim 9 wherein the solubility enhancing tag comprises a glutathione-S-transferase tag, a dihydrofolate reductase tag, a NusA tag or a SNUT tag.

11. The method according to claim 2 wherein the vectors are each transfected or transformed into a plurality of different host cell strains.

12. The method according to claim 2 wherein the host cell strains are different E. coli strains.

13. The method according to claim 12 wherein the E coli strains are selected from Rosetta (DE3) pLacI, Tuner (DE3) pLacI, Origami BL21 (DE3) pLacI and TOP1OF′.

14. The method according to claim 2 including the step of screening transformants for correct orientation of DNA.

15. The method according to claim 14 wherein the step of screening transformants for correct orientation of the insert is performed using dot-blotting.

16. The method according to claim 2 wherein the expression products from said host cells are analysed using ELISA or dot-blotting methods.

17. The method according to claim 2 wherein analysis of expression products includes the use of chloroform and UV light to stain protein on an SDS-PAGE gel.

18. The method according to claim 17, wherein the method further comprises the subsequent use of the chloroform-stained SDS-PAGE gel for western blotting for the identification of proteins.

19. The method according to claim 2 wherein the protein of interest is a protein encoded by the yotiao gene, the murine MAR1 protein or the human Jak1 protein.

20. A method of producing a soluble bioactive domain of a protein of interest comprising the steps:
(a) analysing DNA coding for the protein of interest to identify one or more candidate soluble domains,
(b) providing oligonucleotide primers to amplify DNA encoding each domain,
(c) amplifying said DNA using, in parallel, a standard PCR programme using a predicted annealing temperature for the primers; (ii) a standard PCR programme using a temperature in the range 48 to 52° C., preferably 50° C., as the temperature for annealin; and (iii) a touchdown PCR programme, where the annealing temperature starts at a temperature in the range 62 to 67° C., preferably 65° C., and then gradually decreases to a temperature in the range 48 to 52° C., preferably 50° C., over the subsequent cycles,
(d) cloning amplified DNA from step (b) into a plurality of different expression vectors,
(e) optionally screening clones for correct orientation of DNA,
(f) using each of the vectors of step (d) into which the DNA has been cloned to each transfect or transform a plurality of different host cell strains,
(g) expressing said DNA in one or more of said host cell strains, and
(h) analysing expression products from said host cells for solubility.

21. The method according to claim 20 wherein at least three candidate soluble domains are selected and DNA is amplified for each of said domains.

22. The method according to claim 20 wherein the plurality of vectors include one or more of a vector capable of encoding a fusion protein with a poly-Histidine tag, a vector capable of conferring tight regulation of translation to impose stringent expression conditions, a vector capable of encoding a fusion protein with a solubility enhancing tag.

23. The method according to claim 22 wherein the solubility enhancing tag comprises a glutathione-S-transferase tag, a dihydrofolate reductase tag, a NusA tag or a SNUT tag.

24. The method according to claim 20 wherein the host cell strains are different E. coli strains.

25. The method according to claim 24 wherein the E coli strains are selected from Rosetta (DE3) pLacI, Tuner (DE3) pLacI, Origami B21 (DE3) pLacI and TOP10F.

26. A soluble bioactive domain of a protein produced by the method according to claim 1.

27. Use of a sortase gene product as a purification tag.

28. The use according to claim 27 where in the sortase gene product is a Staphylococcus aureus srtA gene product.

29. The use according to claim 27 wherein the sortase gene product is encoded by the nucleotide sequence shown in FIG. 8 or a variant or fragment thereof.

30. The use according to claim 27 wherein the sortase gene product comprises amino acids 26 to 171 of the SrtA sequence shown in FIG. 8 or a variant or fragment thereof.

31. An expression construct for the production of recombinant polypeptides, which construct comprises an expression cassette consisting of the following elements that are operably linked: a) a promoter; b) the coding region of a DNA encoding a sortase gene product as a purification tag sequence; and c) a cloning site for receiving the coding region for the recombinant polypeptide to be produced; and d) transcription termination signals.

32. The expression construct according to claim 31 wherein the sortase gene product is a Staphylococcus aureus srtA gene product.

33. The expression construct according to claim 31 wherein the sortase gene product is encoded by the nucleotide sequence shown in FIG. 8 or a variant or fragment thereof.

34. The expression construct according to claim 31 wherein the sortase gene product comprises amino acids 26 to 171 of the SrtA sequence shown in FIG. 8 or a variant or fragment thereof.

35. A method for producing a polypeptide, comprising: a) preparing an expression vector for the polypeptide to be produced by cloning the coding sequence for the polypeptide into the cloning site of an expression construct as claimed in claim 30; b) transforming a suitable host cell with the expression construct thus obtained; and c) culturing the host cell under conditions allowing expression of a fusion polypeptide consisting of the amino acid sequence of the purification tag with the amino acid sequence of the polypeptide to be expressed covalently linked thereto; and d) isolating the fusion polypeptide from the host cell or the culture medium by means of binding the fusion polypeptide present therein through the amino acid sequence of the purification tag.

36. The method according to claim 35, wherein the sortase gene product is a Staphylococcus aureus srtA gene product.

37. The method according to claim 35 wherein the sortase gene product is encoded by the nucleotide sequence shown in FIG. 8 or a variant or fragment thereof.

38. The method according to claim 37 wherein the sortase gene product comprises amino acids 26 to 171 of the SrtA sequence shown in FIG. 8 or a variant or fragment thereof.

39. A fusion polypeptide obtained by the method of claim 35.

40. A purification tag comprising a sortase gene product.

41. The purification tag according to claim 40 wherein the gene product is a Staphylococcus aureus srtA gene product.

42. The purification tag according to claim 40 wherein the sortase gene product is encoded by the nucleotide sequence shown in FIG. 8 or a variant or fragment thereof.

43. The purification tag according to claim 40 wherein the sortase gene product comprises amino acids 26 to 171 of the SrtA sequence shown in FIG. 8 or a variant or fragment thereof.
